(19) United States
(12) Patent Application Publication
(10) Pub. No.: US 2021/0317072 A1
(43) Pub. Date: Oct. 14, 2021

(54) H2O CRYSTAL FORMS OF A TCA CYCLE INTERMEDIATE CONJUGATE

(71) Applicant: IMBRIA PHARMACEUTICALS, INC., Boston, MA (US)

(72) Inventor: Yuelie Lu, San Diego, CA (US)

(21) Appl. No.: 17/226,356

(22) Filed: Apr. 9, 2021

Related U.S. Application Data
(60) Provisional application No. 63/008,155, filed on Apr. 10, 2020.

(51) Int. Cl. C07C 229/22; A61K 9/00

(52) U.S. Cl. CPC C07C 229/22; C07B 2200/13; A61K 9/0053

(57) ABSTRACT

The invention provides polymorphs of a compound of Formula (X):

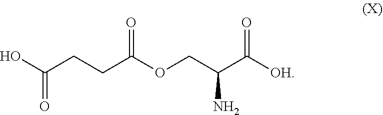 [SMILES: N[C@@H](COC(=O)CCC(=O)O)C(=O)O]

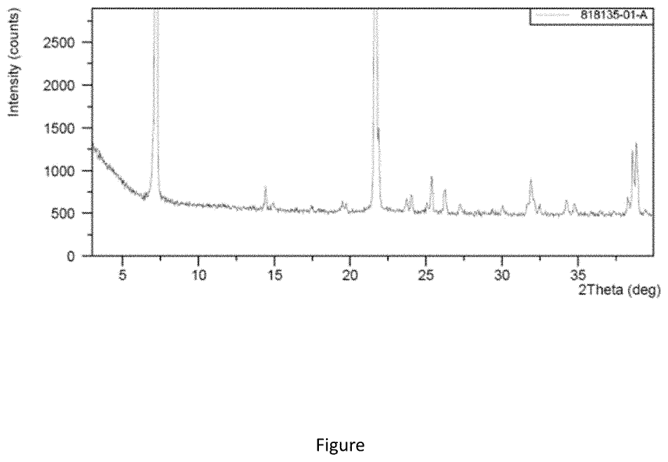

The invention also provided pharmaceutical compositions containing polymorphs of the compound and methods treating conditions in a subject by providing polymorphs of the compound.

### CROSS-REFERENCE TO RELATED APPLICATIONS

This application claims the benefit of, and priority to, U.S. Provisional Application No. 63/008,155, filed Apr. 10, 2020, the contents of which are incorporated herein by reference.

### FIELD OF THE INVENTION

The invention relates to crystallographic forms of a tricarboxylic acid cycle (TCA) intermediate conjugated to an amino acid.

### BACKGROUND

Abnormal metabolism of the tricarboxylic acid (TCA) cycle (also known as the citric acid cycle or Krebs cycle) is associated with a variety of diseases, including inherited metabolic disorders, neurodegenerative diseases, and cancers. Inherited disorders of the TCA cycle cause intellectual disability, various neurological problems, and death in young children, while the neurodegenerative diseases and cancers that are coupled to dysfunction of the TCA cycle lead to cognitive and physical disabilities and death in adults.

Although the TCA cycle and its relationship to other intermediary metabolic pathways have been understood for decades, effective therapies for treating conditions associated with abnormal TCA cycle metabolism are lacking. Efforts to develop compositions that restore TCA cycle metabolism by delivering TCA cycle metabolites have been unsatisfactory. Compounds that provide unadulterated TCA cycle intermediates are challenging to administer orally due to the large amount of material that is needed to be taken by mouth and strong tastes or odors. Existing compositions are inadequate to remedy dysfunction of the TCA cycle, and people continue to suffer and die from a variety of conditions related to abnormal TCA cycle metabolism.

### SUMMARY

It was recently identified that conjugating amino acids to TCA cycle intermediates dramatically increases the solubility of those compounds. By incorporating such intermediates into higher solubility molecules, such molecules can then be metabolized to release the intermediates in the body. Provided herein are crystallographic forms of TCA cycle intermediate conjugates. Specifically, the TCA cycle intermediate conjugate having the structure of a compound of Formula (X):

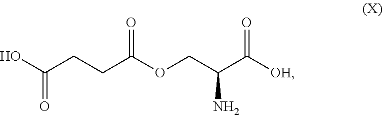 [SMILES: N[C@@H](COC(=O)CCC(=O)O)C(=O)O]

was recently identified as a promising therapeutic candidate for treating conditions associated with abnormal TCA cycle metabolism. The invention recognizes that crystals of compound of Formula (X) exist and that one polymorph, Type A, is the most stable under conditions of ambient temperature and relative humidity. Therefore, Type A crystals of the compound of Formula (X) are useful for the manufacture of pharmaceutical compositions. For example, pharmaceutical compositions that contain the Type A polymorph do not require special handling during storage or distribution. In addition, such compositions may retain their efficacy better than compositions containing other polymorphs or mixtures of polymorphs.

Because the Type A polymorph is water-soluble, it is useful as a therapeutic agent for treating conditions associated with abnormal TCA cycle metabolism. Due to the high solubility of the compounds, they are readily absorbed, circulate throughout the body, and can be cleaved to make the TCA cycle intermediate available to target tissues. In addition, the Type A polymorph is suitable for oral administration because the covalent linkages eliminate the taste or odor produced by free TCA cycle intermediates. Thus the Type A polymorph also results in better patient compliance with a therapeutic regimen compared to formulations that use free TCA cycle intermediates.

The invention also provides methods of treating conditions associated with abnormal TCA cycle metabolism using compounds of Formula (X) polymorphs, such as Type A.

In an aspect, the invention provides crystals comprising a polymorph of a compound of Formula (X):

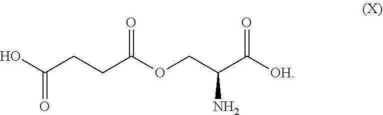 [SMILES: N[C@@H](COC(=O)CCC(=O)O)C(=O)O]

The polymorph may be Type A. The crystal may be substantially free of one or more other polymorphs. For example, the crystal may include a Type A polymorph and be substantially free of any other polymorph.

The crystal may include a hydrated form of the compound of Formula (X). The crystal may include a monohydrate form of the compound. The crystal may include an anhydrous form of the compound.

In another aspect, the invention provides pharmaceutical compositions that include a polymorph of the compound of Formula (X) or a prodrug thereof.

The polymorph may be Type A. The composition may be substantially free of one or more other polymorphs. For example, the composition may include a Type A polymorph and substantially free of any other polymorph.

The composition may include a hydrated form of the compound of Formula (X). The composition may include a monohydrate form of the compound. The composition may include an anhydrous form of the compound.

The composition may be formulated for any route or mode of administration. The composition may be formulated for buccal, dermal, enteral, intraarterial, intramuscular, intraocular, intravenous, nasal, oral, parenteral, pulmonary, rectal, subcutaneous, topical, or transdermal administration. The composition may be formulated for administration by injection or with or on an implantable medical device (e.g., stent or drug-eluting stent or balloon equivalents). Preferably the composition is formulated for oral administration.

The composition may be formulated as a single unit dosage. The composition may be formulated as divided dosages.

The composition may contain a defined dose of the compound. The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

The composition may contain a crystal of the compound of Formula (X). The crystal may have any of the properties described above in relation to crystals of the compound.

In another aspect, the invention provides methods of treating a condition in a subject by providing to a subject having, or at risk of developing, a condition a composition containing a therapeutically effective amount of a polymorph of a compound of Formula (X). The polymorph may be Type A. The composition may have any of the properties described above in relation to compositions that include the compound of Formula (X), including crystals of the compound.

The composition may be provided by any suitable route or mode of administration. The composition may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents). Preferably the composition is provided orally.

The composition may be provided as a single unit dosage. The composition may be provided as divided dosages. The composition may be provided in one dose per day. The composition may be provided in multiple doses per day. The composition may be provided in two, three, four, five, six, eight, or more doses per day.

The composition may contain a defined dose of the compound, such as any of the doses described above. The dose or doses may be provided for a defined period. One or more doses may be provided daily for at least one week, at least two weeks, at least three weeks, at least four weeks, at least six weeks, at least eight weeks, at least ten weeks, at least twelve weeks or more.

The condition may be a condition associated with abnormal TCA cycle metabolism. The condition associated with altered TCA cycle metabolism may be an inherited disorder, such as 2-oxoglutaric aciduria, fumarase deficiency, or succinyl-CoA synthetase efficiency. The condition associated with altered TCA cycle metabolism may be a neurodegenerative disorder, such as Amyotrophic Lateral Sclerosis, Alzheimer's disease, Parkinson's disease, or Huntington's disease. The condition associated with altered TCA cycle metabolism may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma.

The condition associated with altered TCA cycle metabolism may be an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.

### BRIEF DESCRIPTION OF THE DRAWINGS

The FIGURE is an XRPD diffractogram of the Type A polymorph of the compound of Formula (X) starting material.

### DETAILED DESCRIPTION

It has recently been identified that conjugating amino acids to TCA cycle intermediates or prodrugs dramatically increases the solubility of those compounds. The recently-identified compound of Formula (X) holds promise as a therapeutic agent for treating a variety of conditions, including conditions associated with abnormal TCA cycle metabolism: The compound of Formula (X) is a TCA cycle intermediate conjugate with the following structure:

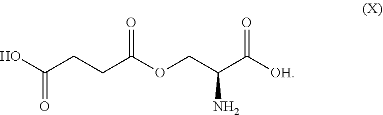 [SMILES: N[C@@H](COC(=O)CCC(=O)O)C(=O)O]

The present invention recognizes that crystals of the compound of Formula (X) exist in multiple polymorphic forms. One polymorph, Type A, is most stable under conditions of ambient temperature and relative humidity and therefore has particular utility for the manufacture of pharmaceutical compositions. Due to the stability of the Type A polymorphs, compositions containing this polymorph can readily be stored and distributed without loss of therapeutic efficacy. Thus, the invention provides compositions containing polymorphs of crystalline compound of Formula (X) or prodrugs thereof, methods of making such compositions, and methods of using them to treat various conditions in a subject.

### Polymorphs of the Compound of Formula (X)

As described in the examples below, crystals of the compound of Formula (X) may exist in the polymorphic Type A.

Crystals may be formed as salts of the compound of Formula (X). For example, crystals may be formed as hydrochloride salts of the compound of Formula (X) or as prodrugs of the compound of Formula (X).

Compounds of the invention may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphrate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, striate, succinate, sulfate, tartrate, thiocyanate, p-toluoenesulfonate, undecanoate, valerate salts, and the like. Representative alkali or alkaline earth metal salts include sodium, lithium, potassium, calcium magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. in some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate, and aryl sulfonate.

### Pharmaceutical Compositions

The invention provides pharmaceutical compositions that contain crystals of a polymorph of the compound of Formula (X) or prodrugs thereof. For example, the composition may contain compound of Formula (X) crystals in Type A. The composition may be substantially free of one or more other polymorphs. For example, the composition may include a Type A polymorph and be substantially free of polymorphs of any other polymorph.

A composition containing a polymorph of Type A may be substantially free of one or more other polymorphic forms of the compound of Formula (X) if the composition contains the predominant polymorph at a defined level of purity. Purity may be expressed as the amount of predominant polymorph as a percentage of the total weight of two of more polymorphs of the compound of Formula (X).

In certain embodiments, the total weight is the weight of all polymorphs of the compound of Formula (X) in the composition. For example, a composition that contains the Type A polymorph and is substantially free of other polymorphs may contain Type A at a defined weight percentage of all polymorphs of the compound of Formula (X) in the composition. For example, the composition may contain Type A at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of all polymorphs of the compound of Formula (X) in the composition.

In certain embodiments, the total weight is the weight of selected polymorphs of the compound of Formula (X) in the composition. For example, a composition that contains the Type A polymorph and is substantially free of any other polymorph may contain Type A at a defined weight percentage of Forms A. For example, the composition may contain Type A at at least 95% by weight, at least 96% by weight, at least 97% by weight, at least 98% by weight, at least 99% by weight, at least 99.5% by weight, at least 99.6% by weight, at least 99.7% by weight, at least 99.8% by weight, or at least 99.9% by weight of Forms A in the composition.

Alternatively or additionally, a composition containing a polymorph of the compound of Formula (X) may be substantially free of one or more other polymorphic forms of the compound of Formula (X) if the composition contains the secondary polymorphs at levels below a defined level. Presence of a secondary polymorphs may be defined as the amount of one or more secondary polymorphs as a percentage of the total weight of two of more polymorphs of the compound of Formula (X).

In certain embodiments, the total weight is the weight of all polymorphs of the compound of Formula (X) in the composition. For example, a composition that contains the Type A polymorph and is substantially free of other polymorphs may contain all polymorphs other than Type A at a defined weight percentage of all polymorphs of the compound of Formula (X) in the composition. For example, the composition may contain all polymorphs other than Type A at below 5% by weight, below 4% by weight, below 3% by weight, below 2% by weight, below 1% by weight, below 0.5% by weight, below 0.4% by weight, below 0.3% by weight, below 0.2% by weight, or below 0.1% by weight of all polymorphs of the compound of Formula (X) in the composition.

In certain embodiments, the total weight is the weight of selected polymorphs of the compound of Formula (X) in the composition. For example, a composition that contains the Type A polymorph and is substantially free of any other polymorph may contain any other polymorph at a defined weight percentage of Forms A and the other polymorphs. For example, the composition may contain the other polymorphs at below 5% by weight, below 4% by weight, below 3% by weight, below 2% by weight, below 1% by weight, below 0.5% by weight, below 0.4% by weight, below 0.3% by weight, below 0.2% by weight, or below 0.1% by weight of Type A and the other polymorph of the compound of Formula (X) in the composition.

The composition may include a hydrated form of the compound of Formula (X). The composition may include a monohydrate form of the compound of Formula (X). The composition may include an anhydrous form of the compound of Formula (X).

The composition may be formulated for any route or mode of administration. The composition may be formulated for buccal, dermal, enteral, intraarterial, intramuscular, intraocular, intravenous, nasal, oral, parenteral, pulmonary, rectal, subcutaneous, topical, or transdermal administration. The composition may be formulated for administration by injection or with or on an implantable medical device (e.g., stent or drug-eluting stent or balloon equivalents). Preferably the composition is formulated for oral administration.

The composition may be formulated as a single unit dosage. The composition may be formulated as divided dosages.

The composition may contain a defined dose of the compound of Formula (X). The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound of Formula (X).

A pharmaceutical composition containing a polymorph of the compound of Formula (X) may be in a form suitable for oral use, such as tablets, troches, lozenges, fast-melts, dispersible powders or granules, or capsules. Compositions intended for oral use may be prepared according to any method known in the art for the manufacture of pharmaceutical compositions and such compositions may contain one or more agents selected from sweetening agents, flavoring agents, coloring agents, and preserving agents, in order to provide pharmaceutically elegant and palatable preparations. Tablets contain the polymorph in admixture with non-toxic pharmaceutically acceptable excipients. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid, or talc. Preparation and administration of pharmaceutical compositions is discussed in U.S. Pat. No. 6,214,841 and U.S. Patent Publication No. 2003/0232877, the contents of each of which are incorporated by reference herein. Formulations for oral use may also be presented as hard gelatin capsules in which the compounds are mixed with an inert solid diluent, such as calcium carbonate, calcium phosphate or kaolin. The formulation may allow controlled release of the polymorph of the compound of Formula (X) in the gastrointestinal tract by encapsulating the polymorph in an enteric coating.

Dispersible powders and granules provide the compounds in admixture with a dispersing or wetting agent, suspending agent and one or more preservatives. Suitable dispersing or wetting agents and suspending agents are exemplified, for example sweetening, flavoring, and coloring agents, may also be present.

The composition may comprise a prodrug of the Type A polymorph of the compound of Formula (X). A prodrug is a medication or compound that, after administration, is metabolized (i.e. converted within the body) into a pharmaceutically active drug. The prodrug itself may be distributed, metabolized, and excreted. The prodrug may improve the bioavailability of the active drug when the active drug is poorly absorbed from the gastrointestinal tract. The prodrug may improve how selectively the drug interacts with cells or processes that are no its intended target, thereby reducing unintended and undesirable side effects. The prodrug may be converted into a biologically active form (bioactivated) inside cells (a Type I prodrug) or outside cells (a Type II prodrug). The prodrug may be bioactivated in the gastrointestinal tract, in systemic circulation, in metabolic tissue other than the target tissue, or in the target tissue. Thus, the compounds of the invention can be metabolized in the body to yield an intermediate of the TCA cycle.

### Providing a Polymorph of the Compound of Formula (X) to a Subject

The invention provides methods of treating a condition in a subject by providing a polymorph of the compound of Formula (X). The polymorph may be Type A. The polymorph of the compound of Formula (X) may be provided in a pharmaceutical composition, as described above. In certain embodiments of the methods, only a polymorph of Type A is provided.

The polymorph of the compound of Formula (X) may be provided by any suitable route or mode of administration. For example and without limitation, the polymorph of the compound of Formula (X) may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents).

The polymorph of the compound of Formula (X) may be provided according to a dosing regimen. A dosing regimen may include a dosage, a dosing frequency, or both.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may contain a defined amount of the compound of Formula (X) that improves cardiac mitochondrial function, such as any of the doses described above in relation to pharmaceutical compositions containing a polymorph of the compound of Formula (X).

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least one week, at least two weeks, at least three weeks, at least four weeks, at least six weeks, at least eight weeks, at least ten weeks, at least twelve weeks or more.

The subject may be a human. The subject may be a human that has a condition associated with abnormal TCA cycle metabolism. The subject may be a human that is at risk of developing a condition associated with abnormal TCA cycle metabolism. A subject may be at risk of developing a condition if the subject does not meet established criteria for diagnosis of the condition but has one or more symptoms, markers, or other factors that indicate the subject is likely to meet the diagnostic criteria for the condition in the future. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

Conditions that May be Treated with a Polymorph of a Compound of Formula (X)

The invention provides methods of treating a condition in a subject by providing a polymorph of a compound of Formula (X). The condition may be any disease, disorder, or condition for which increasing mitochondrial energy production provides a therapeutic benefit.

The condition may be a condition associated with abnormal TCA cycle metabolism. For example and without limitation, the condition associated with altered TCA cycle metabolism may be an inherited disorder, such as 2-oxoglutaric aciduria, fumarase deficiency, or succinyl-CoA synthetase efficiency. The condition associated with altered TCA cycle metabolism may be a neurodegenerative disorder, such as Amyotrophic Lateral Sclerosis, Alzheimer's disease, Parkinson's disease, or Huntington's disease. The condition associated with altered TCA cycle metabolism may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma.

The condition associated with altered TCA cycle metabolism may be an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.

### EXAMPLES

### Example 1

Summary

A comprehensive polymorph screening for a TCA intermediate conjugated to an amino acid which has the structure of Formula (X) was undertaken. The TCA conjugate starting material was characterized by X-ray powder diffraction (XRPD). The data showed that the material is crystalline in nature and has similar XRPD pattern to that of the Type A. Starting with Type A, polymorph/single crystal screening experiments were set up under 32 conditions using methods of solid vapor diffusion, slurry conversion at room temperature, slurry conversion at 50° C., and temperature cycling. One unique XRPD patterns was observed, which was Type A. Type A is a stable Type At the ambient temperature and humidity.

Characterization of Type A

The starting material of the compound of Formula (X) was characterized using X-ray powder diffraction (XRPD).

The FIGURE is an XRPD diffractogram of the compound of Formula (X) starting material. The XRPD results suggested high crystallinity of the starting material.

X-ray powder diffraction data has been challenging to interpret due to the extreme preferred orientation, which results in large variations in the peak intensities from one sample preparation to the next. To minimize this effect, single crystal X-ray diffraction was used to acquire the crystallographic structure, and the X-ray powder diffraction that should be observed in an ideal sample that is absent of preferred orientation was calculated.

Polymorph/Single Crystal Screening

For with Type A, polymorph screening experiments were set up under 32 conditions using methods of solid vapor diffusion, slurry conversion at room temperature, slurry conversion at 50° C., and temperature cycling. Polymorph screening experiments were performed using different crystallization or solid transition methods. Polymorph screening experiments are summarized in Table 1.

TABLE 1

| | No. of | |
|---|---|---|
| Method | Experiments | Crystal Type |
| Temperature cycling | 6 | Type A, Type A + peak |
| Slurry at room temperature | 11 | Type A, Type A + peak |
| Slurry at 50° C. | 9 | Type A, Type A + peak |
| Solid vapor diffusion | 6 | Type A |
| Total | 32 | Type A, Type A + peak |

Slurry conversion experiments were conducted at room temperate and at 50° C. in different solvent systems. Solids were isolated for XRPD analysis. Results from slurry conversion experiments are summarized in Table 2 and Table 3.

TABLE 2
Slurry experiments for starting material
(818135-04-A) at room temperature

| Experiment ID | Solvent | Solid Form |
|---|---|---|
| 818135-04-A1 | MeOH | Type A |
| 818135-04-A2 | Acetone | Type A |
| 818135-04-A3 | IPAc | Type A + peak |
| 818135-04-A4 | MTBE | Type A + peak |
| 818135-04-A5 | CAN | Type A + peak |
| 818135-04-A6 | DCM | Type A + peak |
| 818135-04-A7 | H₂O | Type A |
| 818135-04-A8 | THF/H₂O (0.98:0.02, a_{W}~0.2) | Type A |
| 818135-04-A9 | THF/H₂O (0.956:0.044, a_{W}~0.4) | Type A + peak |
| 818135-04-A10 | THF/H₂O (0.924:0.076, a_{W}~0.6) | Type A |
| 818135-04-A11 | THF/H₂O (0.87:0.13, a_{W}~0.8) | Type A |

TABLE 2
Slurry experiments for starting material (818135-05-A) at 50° C.

| Experiment ID | Solvent | Solid Form |
|---|---|---|
| 818135-05-A1 | EtOH | Type A |
| 818135-05-A2 | EtOAc | Type A |
| 818135-05-A3 | 2-Me THF | Type A |
| 818135-05-A4 | n-heptane | Type A + peak |
| 818135-05-A5 | MEK | Type A |
| 818135-05-A6 | 1,4-dioxane | Type A |
| 818135-05-A7 | CHCL₃ | Type A |
| 818135-05-A8 | DMSO | On-going |
| 818135-05-A9 | Toluene | Type A |

Solid vapor diffusion experiments were conducted in different solvent conditions. The precipitates were isolated for XRPD analysis. Results from solid vapor diffusion experiments are summarized in Table 4.

TABLE 4
Solid Vapor Diffusion Experiments and solid
form of starting material (818135-06-A)

| Experiment ID | Anti-solvent | Solid Form |
|---|---|---|
| 818135-06-A1 | DCM | Type A |
| 818135-06-A2 | EtOH | Type A |
| 818135-06-A3 | THF | Type A |
| 818135-06-A4 | DMSO | Type A |
| 818135-06-A5 | Acetone | Type A |
| 818135-06-A6 | H₂O | Type A |

### CONCLUSIONS

The Type A was successfully characterized to understand its form behavior. A comprehensive polymorph screening in 32 different conditions was performed. The polymorph of the compound of Formula (X) was identified during the screening, specifically Type A

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A crystal comprising a polymorph of a compound of Formula (X): 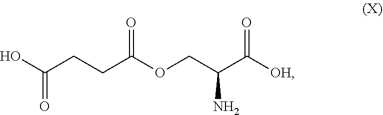 [SMILES: N[C@@H](COC(=O)CCC(=O)O)C(=O)O]
wherein the crystal is a Type A polymorph of a compound of Formula (X).

2. The crystal of claim 1, wherein the crystal comprises a salt of the compound.

3. The crystal of claim 2, wherein the crystal comprises a hydrated form of the compound.

4. The crystal of claim 3, wherein the hydrated form of the compound is monohydrate.

5. The crystal of claim 1, wherein the crystal is substantially free of other polymorphs of the compound of Formula (X).

6. A pharmaceutical composition comprising a Type A polymorph of a compound of Formula (X):

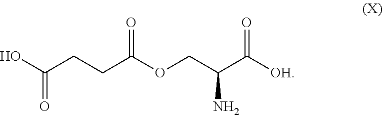 [SMILES: N[C@@H](COC(=O)CCC(=O)O)C(=O)O]

7. The composition of claim 6, wherein the composition comprises a salt of the compound.

8. The composition of claim 7, wherein the composition comprises a hydrated form of the compound.

9. The composition of claim 8, wherein the hydrated form of the compound is monohydrate.

10. The composition of claim 6, wherein the composition is substantially free of other polymorphs the compound of Formula (X).

11. The composition of claim 6, wherein the composition is formulated for oral administration.

12. The composition of claim 6, wherein the composition is formulated as a single unit dosage.

13. The composition of claim 6, wherein the composition is formulated as divided dosages.

14. A method of treating a condition in a subject, the method comprising providing to a subject having, or at risk of developing, a condition a composition comprising a therapeutically effective amount of a Type A polymorph of a compound of Formula (X):

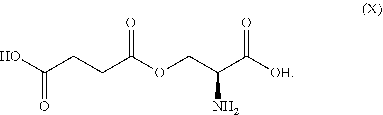 [SMILES: N[C@@H](COC(=O)CCC(=O)O)C(=O)O]

15. The method of claim 14, wherein the composition comprises a salt of the compound.

16. The method of claim 15, wherein the composition comprises a hydrated form of the compound.

17. The method of claim 16, wherein the hydrated form of the compound is monohydrate.

18. The method of claim 14, wherein the composition is provided orally.

19. The method of claim 14, wherein the condition is a condition associated with abnormal TCA cycle metabolism.

20. The method of claim 19, wherein the condition is an inherited disorder, 2-oxoglutaric aciduria, fumarase deficiency, succinyl-CoA synthetase efficiency, a neurodegenerative disorder, Amyotrophic Lateral Sclerosis, Alzheimer's disease, Parkinson's disease, Huntington's disease, a cancer, pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, neuroblastoma, an energetic disorder, refractory epilepsy, propionic academia (PA), methylmalonic academia (MMA), a long chain fatty acid oxidation disorder, succinyl CoA lyase deficiency, pyruvate carboxylase deficiency, mitochondrial respiratory chain deficiency, glutaric academia type 1 or type 2 a neurological disease, disorder or condition, a pain or fatigue disease, muscular dystrophy (e.g. Duchenne's muscular dystrophy, and Becker's muscular dystrophy), mitochondrial myopathy, mitochondrial encephalomyopathy lactic acidosis and stroke-like syndrome (MELAS), myoclonic epilepsy and ragged-red fibers (MERRF), a mitochondrial associated disease, or a disorder related to POLG mutation.
